(19) United States
(12) Patent Application Publication
(10) Pub. No.: US 2009/0126454 A1
(43) Pub. Date: May 21, 2009

(54) Method of Measuring a Target Gas at Low Concentration

(76) Inventors: Keith Pratt, Portsmouth (GB); Peter Smith, Yarnton (GB)

Correspondence Address: HONEYWELL INTERNATIONAL INC., 101 COLUMBIA ROAD, P O BOX 2245, MORRISTOWN, NJ, 07962-2245, US

(21) Appl. No.: 11/954,523

(22) Filed: Dec. 12, 2007

(30) Foreign Application Priority Data
Nov. 16, 2007 (GB) 0722574.1

(51) Int. Cl. G01N 37/00; G01N 33/00

(52) U.S. Cl. 73/1.02; 73/23.2

(57) ABSTRACT

A method for determining the concentration of a target gas in an environment which utilises a metal oxide gas sensor having a sensitivity to a background stimulus present in the environment, said sensitivity being variable according to the concentration of the target gas, comprises the following steps:
(1) calibrating the sensitivity of the first sensor to the background stimulus as a function of the concentration of the target gas;
(2) determining a change in the sensitivity of the first sensor to the background stimulus in the environment, caused by the presence of the target gas in the environment; and
(3) using the change in sensitivity to the background stimulus determined in step (2) and the step of calibrating the sensitivity of the first sensor in step (1) to determine the concentration of the target gas in the environment.

### FIELD OF THE INVENTION

The present invention relates to a method for measuring a low concentration of a target gas in the presence of a much higher concentration of an interferent gas.

### BACKGROUND OF THE INVENTION

It is often desirable to be able to measure a low concentration of a target gas in is the presence of a much higher concentration of an interferent gas. One specific example of this is the measurement of hydrogen sulphide in reformate gas used in fuel cells. Typical conditions experienced in this type of environment are high gas temperature, ppm or ppb levels of hydrogen sulphide, low oxygen concentration and high concentration of hydrogen (e.g. 45 vol. %). Both the oxygen and hydrogen concentrations may be fluctuating. The high temperature environment prevents the use of conventional ‘wet’ electrochemical sensors for hydrogen sulphide, which in any case still have sufficiently high hydrogen cross-sensitivity to be unusable. Heated semiconducting metal oxide sensors can operate in this type of environment, but all known metal oxide sensors have cross-sensitivity to hydrogen. For example, metal oxide sensors based on chromium titanate have a similar magnitude of response to 10 ppm hydrogen sulphide and 500 ppm hydrogen. Thus, in the presence of a fluctuating background of hydrogen at vol. % levels, the signal due to a few ppm hydrogen sulphide would be too small to measure. The effect is made worse by the fact that the signals to both gases are dependent on the oxygen and water content of the gas, both of which may also be fluctuating.

Many metal oxide hydrogen sulphide sensors, particularly those based on tungsten trioxide, also suffer from an effect whereby they lose sensitivity if not frequently exposed to hydrogen sulphide. This effect is known in the trade as the sensor ‘going to sleep’.

### SUMMARY OF THE INVENTION

Certain gases, including hydrogen sulphide, give rise to a ‘conditioning’ effect on semiconducting metal oxide gas sensors. Conditioning can result in a temporary change in sensitivity of the sensor both to the conditioning gas itself and also to a background stimulus, typically another gas, or mixture of other gases, which may be present in the environment. The invention makes use of these changes in sensitivity. By way of example, if the concentration of the other, interferent gas(es), is known, or can be independently measured, then the magnitude of the change in sensitivity of the conditioned sensor to the interferent gas(es) can be used to calculate the concentration of the conditioning gas. Thus, the concentration of the conditioning gas can be measured indirectly.

According to a first aspect of the present invention, therefore, a method is provided for determining the concentration of a target, conditioning, gas in an environment which utilises a first metal oxide sensor having a sensitivity to a background stimulus in the environment, said sensitivity being variable according to the concentration of the target gas in the environment, the method comprising the following steps:
(1) calibrating the sensitivity of the first sensor to the background stimulus as a function of the concentration of the target gas;

(2) determining a change in the sensitivity of the first sensor to the background stimulus in the environment, caused by the presence of the target gas in the environment; and

(3) using the change in sensitivity to the background stimulus determined in step (2) and the step of calibrating the sensitivity of the first sensor in step (1) to determine the concentration of the target gas in the environment.

### BRIEF DESCRIPTION OF THE DRAWING

FIG. 1 plots sensor response (R/R₀) and concentration of hydrogen and hydrogen sulphide, with time, where R is the resistance of the sensor and R₀ is the baseline resistance of the sensor, ie. the resistance of the “unconditioned” sensor in clean air, without target, or conditioning, gas.

FIG. 2 plots sensor response (R/R₀) and concentration of hydrogen and hydrogen sulphide with time, with temperature cycling of the sensor between heated temperatures of 517° C. and 702° C. for 5 minutes at each temperature.

FIG. 3 plots data from FIG. 2, expressed as sensor response shortly after dropping to 517° C. (Rₛₜₐᵣₜ/R₀), ie. within approximately 30 seconds, and the ratio of the sensor response to hydrogen at the end of the low temperature cycle divided by this initial sensor response (R_{end}/Rₛₜₐᵣₜ), with time.

FIG. 4 plots sensor response to hydrogen sulphide (R/R₀) in the absence of hydrogen; sensor response to hydrogen in the absence of hydrogen sulphide; and concentration of both hydrogen and hydrogen sulphide, with time in temperature cycling mode as per FIG. 2.

FIG. 5 plots sensor response in temperature cycling mode as per FIG. 2, and concentration of hydrogen and hydrogen sulphide, with time.

### DETAILED DESCRIPTION OF THE INVENTION

The invention requires the target gas to be a conditioning gas, ie. a gas which changes the properties, and thus the sensitivity, of the sensing material (from which the sensor is made) in a reversible way. This particularly applies to sulphur-containing gases, and in particular hydrogen sulphide, but may well be applicable to other gases. The effect of hydrogen sulphide on metal oxide sensors is two-fold: firstly there is a direct response due to rapid reversible adsorption and/or chemical reaction; and secondly there is a conditioning effect whereby the gas sensing material is chemically modified by the gas. In the case of hydrogen sulphide, X-ray photoelectron spectroscopy measurements have shown that following exposure the surface of the sensing material contains sulphur groups and hydroxyl groups; see Dawson et al, Sensors and Actuators B: Chemical, Volume 26, Issues 1-3, May 1995, pages 76-80.

The effect of the conditioning varies with gas sensing material and operating temperature, but typically gives rise to a change in baseline resistance and/or a change in response to the conditioning gas after exposure to said gas. The effect is time dependent, ie. exposure to a certain concentration of the conditioning gas for a certain time produces a certain level of conditioning. Continued exposure beyond a certain time typically does not cause further change due to an equilibrium ‘conditioning’ level being reached. Over time, the conditioning effect wears off. The effect is lessened, or cancelled altogether, at elevated temperatures, and embodiments of the present invention make use of this, as will be described in detail below.

The conditioning effect is particularly well known for hydrogen sulphide sensors based on tungsten trioxide. Thus, tungsten trioxide sensors find particular use in the present invention. Tungsten trioxide sensors are known to ‘go to sleep’ if not frequently exposed to hydrogen sulphide. This is because clean, unconditioned, tungsten trioxide has a low sensitivity to hydrogen sulphide, but following conditioning (exposure to the gas for a short length of time) its sensitivity is greatly enhanced. Over time the conditioning wears off. It should be noted, however, that ‘conditioning’ as used in the context of the present invention is not the same as ‘poisoning’, the latter being irreversible and typically caused by silicon-containing species.

The conditioning effect is not specific to tungsten oxide but also occurs with other metal oxides, such as tin oxide and chromium titanate, and thus these, and other, sensing materials are also suitable for use in the present invention. Chromium titanate is the preferred sensing material.

Conversely, if a sensor is used for detecting a gas such as hydrogen sulphide and it has not been initially conditioned by sufficient exposure to the target gas, then its calibrated sensitivity will be artificially low, if insufficient time has elapsed to condition the sensor. Then, on subsequent operation when exposed to the target gas the sensitivity will increase over time with the result that the sensor will exhibit an upward drift in signal. The effect may also manifest itself on the sensor baseline resistance, since this can also change with conditioning. The net result is that the sensor appears to be sluggish, unstable and/or drifting over time.

It has now been found that conditioning by gases such as hydrogen sulphide can also affect the response of the sensing material to a background stimulus, typically in the form of another gas or mixtures of gases. This may be due to the increased level of hydroxylation of the sensing material (which is much higher than can be achieved by exposure to humidity alone) and/or to the presence of sulphur species on the surface of the sensing material which can themselves act in a similar way to chemisorbed oxygen species which are normally responsible for the gas response.

The invention, therefore, measures the change in sensitivity to such a background stimulus which is a result of conditioning by the target gas. Implementation of this requires that the level of conditioning achieved be related to the background stimulus in a known way. The first step, therefore, is to calibrate the sensitivity of the sensor to the background stimulus as a function of the concentration of the target gas.

Then, it is necessary to determine the change in sensitivity of the sensor to the background stimulus caused by the presence of the target gas. This, together with the initial calibration step, can then be used to infer the concentration of the target gas.

In situations where a change in the background stimulus substantially affects the sensitivity of the sensor to the target gas, the method of the present invention might not be as accurate as is desired. However, in such situations a further calibration step may be helpful, to calibrate the sensitivity of the sensor to the background stimulus in the presence of the target gas as a function of how the background stimulus is varying, for instance as a function of concentration in the context of an interferent gas.

As mentioned above, typically the background stimulus will be an interferent gas, or mixture of interferent gases. As a result, the present invention is described herein primarily on this basis. In the case of a mixture of interferent gases, typically one of the interferent gases will be present in substantially higher concentration than the other(s), and vastly in excess of the concentration of the target gas. In such situations, variations in concentration of the interferent gas(es) present in lower concentration will likely not have much effect, if any, on the sensitivity of the method of the claimed invention in determining the concentration of the target gas. The simple situation is that in which the interferent gas, or mixture of interferent gases, is present in the environment of interest at a substantially constant concentration, such that any small variation in concentration or mixture proportions does not substantially affect the sensitivity of the sensor thereto. However, where there is a change in concentration that might affect the gas sensitivity of the sensor, eg. where the oxygen and/or humidity content of the gas is varying, an extra calibration step may be performed, as described above.

It is desirable to reset the sensitivity of the sensor to the background stimulus to its sensitivity prior to exposure to the target gas, before taking further measurements. This is achieved by subjecting the sensor to elevated temperature to substantially remove the ‘conditioning’ effect of the target gas on the sensitivity of the sensor, ie. by subjecting the sensor to a ‘purge’ temperature. Indeed, resetting of the sensitivity of the sensor may be achieved by cycling between elevated and lower temperature conditions, either substantially continuously or intermittently.

Where there is substantially no change in concentration of the interferent gas, or interferent gas mixture, in one embodiment the method of the present invention requires that the concentration of the interferent gas(es) in the environment should be known or determined by independent means, so that the change in sensitivity to that known amount of interferent gas(es) can then be used, together with the initial calibration step mentioned above, to determine the concentration of the target gas.

There are various ways of determining the concentration of the interferent gas(es) in the environment of interest.

One method involves use of the sensor at a high “purge” temperature effective to remove target gas present in or on the sensor, to give an accurate measurement of the interferent gas(es), prior to determining any change in sensitivity due to the presence of the target gas.

Another method involves use of another, or second, sensor maintained in the environment at a high ‘purge’ temperature, or temperature cycled between a high “purge” temperature and lower temperature, thereby maintaining the second sensor in an unconditioned state, while the first sensor operates continuously. The difference in signals obtained from the first and second sensors can then be used to infer the concentration of the conditioning gas. The second sensor should ideally be of the same general type of sensing material as the first sensor, and should operate at substantially the same operating temperature.

It may not be necessary to take measurements under different temperature conditions, as it is known that exposure of certain metal oxides which have been conditioned by hydrogen sulphide to certain reducing gases results in ‘cleaning up’ of the sensor due to reaction of the reducing gas with the modified surface; see Pratt et al, Sensors and Actuators B:Chemical Volume 45 Issue 2, December 1997, p. 147-153. Therefore, another method of determining the concentration of the interferent gas(es) is to use a second sensor, ideally of the same general type of sensing material and operating at substantially the same operating temperature as the first sensor, which is subjected to intermittent flushing with a gas which removes the conditioning gas, provided that this gas does not affect the ability of the sensor to determine the concentrations of the interferent gas(es). The gas used for flushing the second sensor may be a reducing gas, for instance, methane, but this is not essential. The gas may instead be, for instance, ozone or simply clean air.

Yet another method for determining the concentration of the interferent gas(es) is to use a second sensor, again ideally of the same general type of sensing material and operating at substantially the same operating temperature as the first sensor, but which is protected from conditioning by, for instance, a suitable external filter or a layer deposited on the sensor which selectively excludes the conditioning gas,

Yet another method of determining the amount of the interferent gas is to use a second sensor of a different sensing material to the first sensor, and which has a sensitivity to the interferent gas which is not affected by the presence of the target gas.

However, none of the above-described approaches using a second sensor is ideal, as this adds to cost and complexity of design.

Yet another option is to use a different approach to measurement of the concentration of the interferent gas(es) altogether, ie. not to rely upon use of a metal oxide or other sensor.

Alternatively, no independent measurement of the amount of the interferent gas(es) may be necessary, where for instance its concentration is known or may be determined from process conditions used to make the interferent gas(es).

There are, however, embodiments of the present invention which do not require independent measurement of the concentration of the interferent gas(es) or even knowledge of this concentration. Instead, all that is necessary is that the change in sensitivity of the first sensor to the interferent gas(es) be determined.

For instance, this may be achieved by cycling the first sensor temperature between a ‘measuring’ temperature and a higher ‘purge’ temperature, whereby the ‘purge’ temperature and the time at this ‘purge’ temperature is sufficient to substantially cancel the conditioning effect due to the target gas. Then, on initially dropping to the measuring temperature (ie. shortly after treatment at the ‘purge’ temperature) the sensor signal will be that due to the background stimulus, in the absence of conditioning. The ‘measuring’ temperature will vary according to the environment under consideration, but will be a temperature at which conditioning takes place.

In the context of the present invention, shortly after treatment at the ‘purge’ temperature typically means within sufficient time for the sensor to have stabilised but not to have become significantly conditioned. The extent of stabilisation and conditioning which are acceptable for this ‘unconditioned’ reading depends on the required accuracy of the resulting measurement. Typically, this will take a matter of a few minutes, more typically less than a minute, eg. approximately 30 seconds or less.

After a longer period of time at the measuring temperature, the sensor will be modified by conditioning, and will ultimately reach a ‘fully’ conditioned state. The difference in the two signals, ie. shortly after treatment at the ‘purge’ temperature and at the ‘measuring’ temperature, can then be used, together with the calibration performed earlier (ie. of sensor sensitivity to the background stimulus as a function of the concentration of the target gas), to infer the concentration of the target, conditioning, gas. It is not necessary for the two signals to correspond to completely ‘clean’ and fully ‘conditioned’ states, provided that there is sufficient repeatability and distinction between the two states.

An alternative approach could be to measure the sensor signal at the high (or ‘purge’) temperature itself, ie. without dropping to the ‘measuring’ temperature, and then at the lower ‘measuring’ temperature, whereby the high temperature gives the ‘clean’ response and the low temperature, after a known time, gives the ‘conditioned’ response. A disadvantage of this latter approach, however, is that the relative cross-sensitivities to different gases at the two temperatures may be different, causing complications especially where multiple interferent gases or a variable oxygen background is present.

The frequency with which temperature cycling is performed will vary according to the nature of the conditioning gas, and in particular how easy or difficult it is to remove the conditioning effect, and how quickly reconditioning occurs. Typically, however, temperature cycling will involve maintaining the sensor at each of the two temperatures for just minutes, or perhaps tens of seconds, or perhaps for even a shorter period of time. Temperature cycling be performed continuously or intermittently.

It may also be desirable to take additional readings during the low temperature ‘measuring’ phase to deconvolute the transient responses of the interferent gas(es) alone and the slower transient nature of the conditioning.

A number of other approaches can be considered whereby some form of temperature modulation is applied and the resulting signal is analysed, for example the use of a sinusoidally varying temperature at a constant or variable frequency with appropriate signal processing.

Since, in accordance with these aspects, the invention makes direct use of, and controls, the conditioning of the sensor, by application of temperature cycling it also overcomes the issue of metal oxide hydrogen sulphide sensors ‘going to sleep’, since this effect itself is due to conditioning effects wearing off in an uncontrolled manner.

The method of the present invention is particularly applicable to the measurement of low concentrations of hydrogen sulphide (a target, or conditioning, gas) in the presence of high concentrations of hydrogen (an interferent gas), for instance in applications such as fuel cell reformate gas.

Other potential end applications include sour gas monitoring (eg. in oilfields), where it is necessary to measure simultaneously a flammable gas, such as methane (as the interferent gas) and a low level of hydrogen sulphide (as the target gas).

According to second and third aspects of the present invention, use may be made of the discovery that the effect of a target, conditioning, gas on a gas sensing material may be removed by temperature modulation, in order to overcome the phenomenon of a sensor “going to sleep” after exposure to a target, conditioning gas, and after the conditioning effect wears off. In this situation, on subsequent exposure to the target gas, the response, or sensitivity, of the sensor is lower than what it should have been. In the past, use has been made of additional target gas to “wake-up” the sensor and to reset its sensitivity.

The second aspect of the present invention offers an alternative to this, and in particular provides a method of resetting the sensitivity of a metal oxide gas sensor which has an increased or decreased sensitivity to a target gas as a result of exposure to the target gas, to the sensitivity to the target gas prior to exposure to the target gas, the method comprising subjecting the sensor to elevated temperature to substantially remove the ‘conditioning’ effect of the target gas on the sensitivity of the sensor.

The third aspect of the present invention provides a method of resetting the sensitivity of a metal oxide gas sensor which has an increased sensitivity to an interferent gas as a result of exposure of the sensor to a target gas to the sensitivity to said interferent gas prior to exposure to the target gas, the method comprising subjecting the sensor to elevated temperature to substantially remove the ‘conditioning’ effect of target on the sensitivity of the sensor.

Typically, the methods according to the second and third aspects of the present invention involve cycling the sensor between an elevated temperature and a lower temperature, both determined by the nature of the sensor itself. These methods find particular use in relation to hydrogen sulphide sensors, most particularly those used in oil field applications.

The invention is now further illustrated by way of the following Example.

### EXAMPLE

The sensor used was a Capteur model CAP25, manufactured by City Technology Ltd. The sensing material was chromium titanate, which has, by metal oxide sensor standards, a particularly good selectivity to hydrogen sulphide. A sensitivity to hydrogen sulphide of approximately 20 times that to hydrogen is typical.

Experiments were performed using an in-house constructed computer controlled test rig, with provision to cycle the sensor heater between two known temperatures, and the ability to mix wet air, dry air and two gases using mass flow controllers.

All experiments were performed in a background of 21% oxygen, balance nitrogen, at nominally 25% relative humidity.

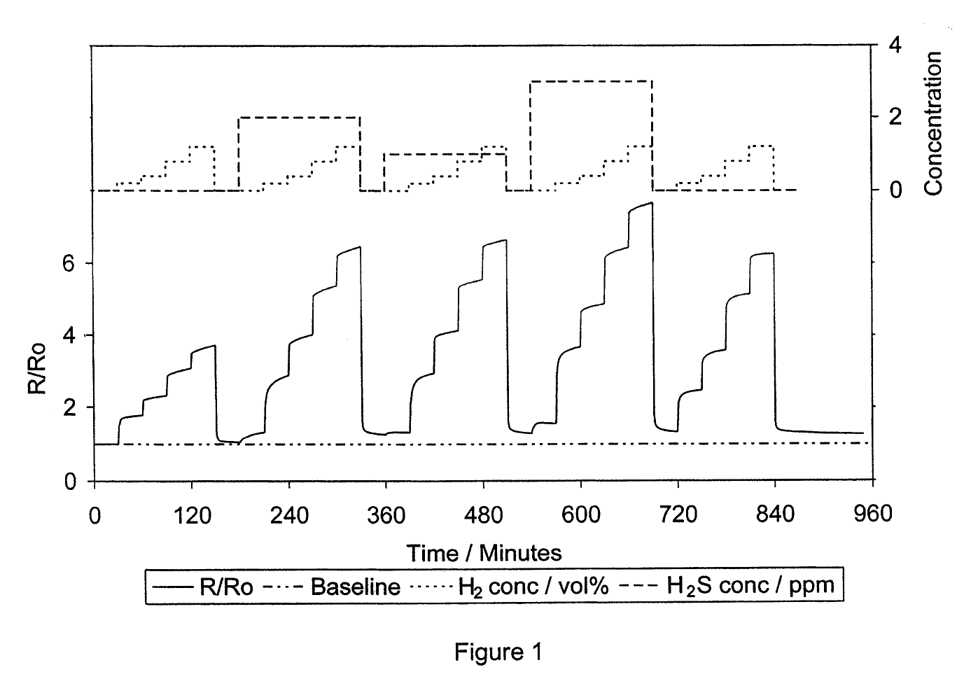

In interpreting the results obtained, reference is made to the accompanying FIGS. 1-5 which were briefly described above.

In more detail, FIG. 1 shows the response of the sensor to repeated exposure to four increasing concentrations of hydrogen, in a background of differing hydrogen sulphide concentrations. Note the different scales for the concentrations for the two gases. It is particularly noticeable from this Figure that the sensitivity to hydrogen during and after exposure to hydrogen sulphide is significantly greater than that before exposure. However, it can also be seen that varying the hydrogen sulphide concentration has relatively little effect on the hydrogen sensitivity. In particular, the sensitivity to the third set of hydrogen exposures is slightly higher than the second set, even though the hydrogen sulphide concentration is lower. Also, the sensitivity to the fifth set of hydrogen exposures is still almost as high as the second and third, even though hydrogen sulphide is no longer present. Both of these effects can be explained by the fact that conditioning takes a long time to wear off at this sensor temperature, so that once the sensor has been conditioned the hydrogen sensitivity remains enhanced.

Using the sensor in this manner, it would therefore be possible only to give a semiquantitative measure of the highest hydrogen sulphide concentration to which the sensor had been previously exposed. Also, as the conditioning effect itself is fairly slow, the signal would be dependent to some extent on the length of exposure, saturating after a certain dosage. For some applications this might be acceptable, for example the system being protected by the sensor may be able to tolerate a certain dosage of maximum concentration of hydrogen sulphide. Ideally, however, it would be desirable to obtain a real time measure of the gas concentration, unaffected by the history of the sensor.

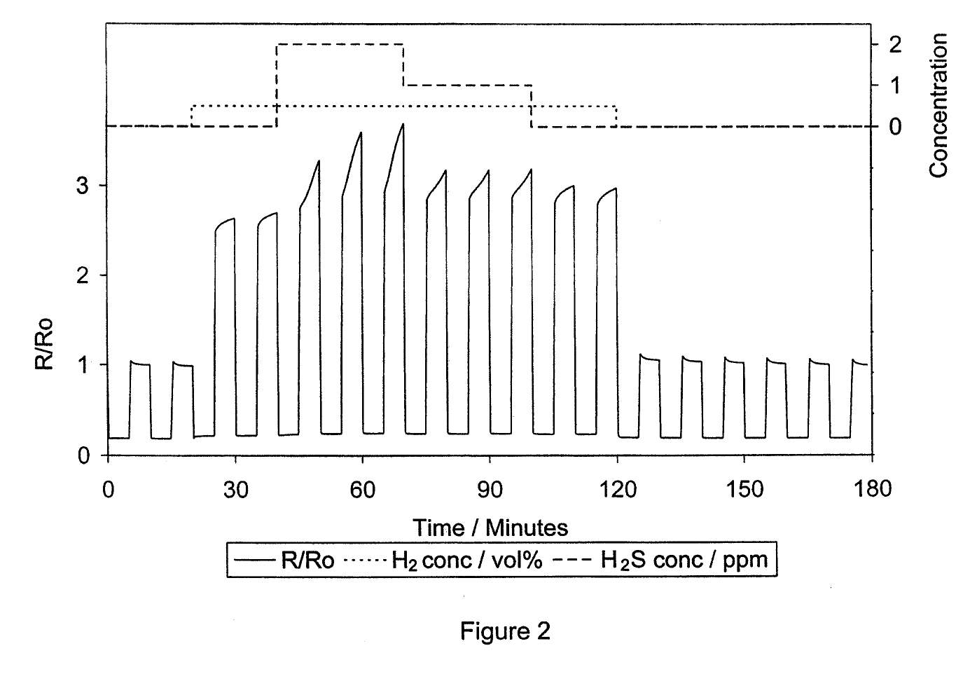

FIG. 2 shows the behaviour of the same sensor using a temperature cycling approach, cycling between sensor heater temperatures of 517° C. and 702° C. for 5 minutes at each temperature. The regions where the sensor resistance is very low (R/Ro<<1) are where the heater is at the high ‘purge’ temperature (ie. 702° C.). On switching to the lower ‘measuring’ temperature (ie. 517° C.), in clean air as shown by the first two temperature cycles, apart from a small overshoot, the sensor resistance remains at the baseline value (R/Ro=1). It should also be noted that on returning to clean air following the hydrogen sulphide and hydrogen exposures, at time>120 minutes, the low temperature transients recover immediately to the baseline value, showing that the high temperature cycle is sufficient to ‘purge’ the sensor of any residual conditioning. This can be compared with the behaviour in FIG. 1, where at the end of the experiment a residual upwards shift in baseline is still apparent.

The effect of hydrogen sulphide on the hydrogen response is very clear in FIG. 2. The transient response changes from a small convex transient (ie. approaching a plateau) in the absence of hydrogen sulphide, to a steep concave transient in the presence of hydrogen sulphide. Changing from 2 ppm to 1 ppm has a significant effect, and the effect is reversible.

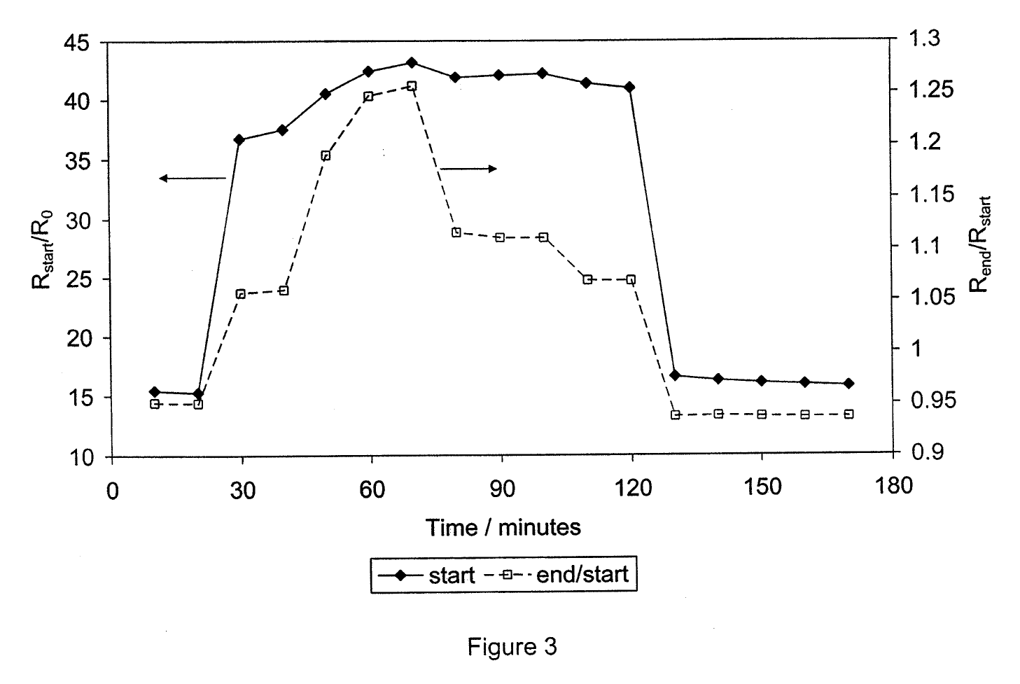

The data in FIG. 2 has been processed in FIG. 3 by separating out the initial and transient behaviour of each low temperature cycle. The initial response data (actually 30 seconds after switching from the high temperature to allow settling) expressed as Rₛₜₐᵣₜ/R₀, is relatively unaffected by the presence of hydrogen sulphide as the sensor has just been purged and has not been exposed to hydrogen sulphide for long enough to cause significant conditioning. Thus the initial response gives a signal strongly dependent on hydrogen concentration but relatively independent of hydrogen sulphide concentration. Conversely, the sensor response at the end of the low temperature cycle, R_{end}/R₀, is strongly dependent on hydrogen sulphide. Taking the ratio of end response over initial response, R_{end}/Rₛₜₐᵣₜ as shown in FIG. 3, allows better separation of the responses to the two gases.

This work demonstrates the feasibility of the approach embodied by the present invention, and suitable methods of resolving/processing the data obtained are well known to those skilled in the art.

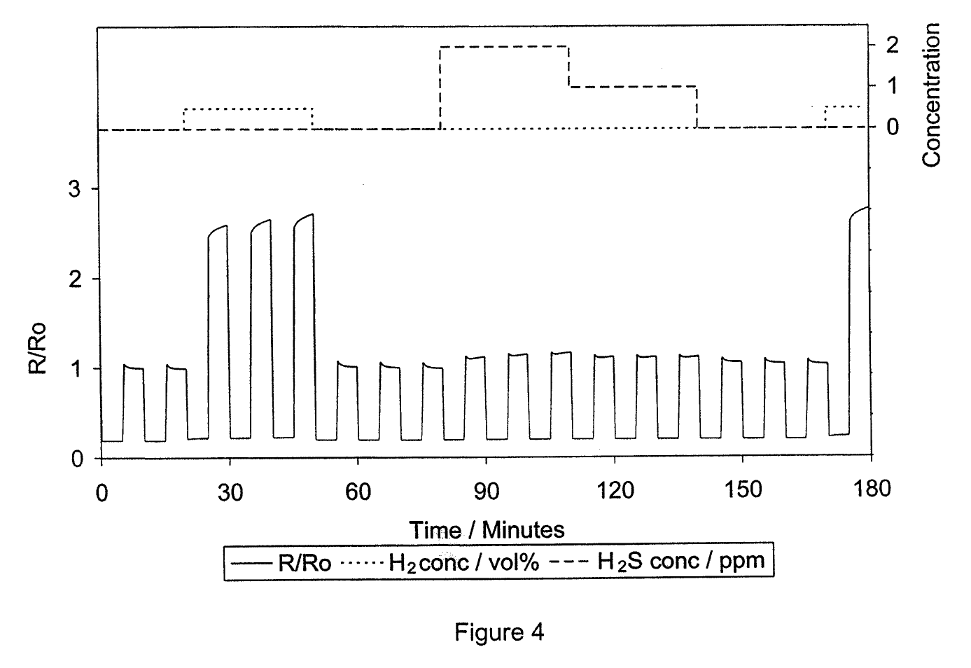

FIG. 4 shows the response of the sensor in temperature cycling mode to the same concentrations of hydrogen sulphide as in FIG. 2, but in the absence of hydrogen. FIG. 4 also shows the response of the sensor to hydrogen in the absence of hydrogen sulphide. It is clear from visual inspection of FIGS. 2 and 4 that the effect of hydrogen sulphide is greatly enhanced by the presence of hydrogen. This clearly demonstrates the main principle of the present invention.

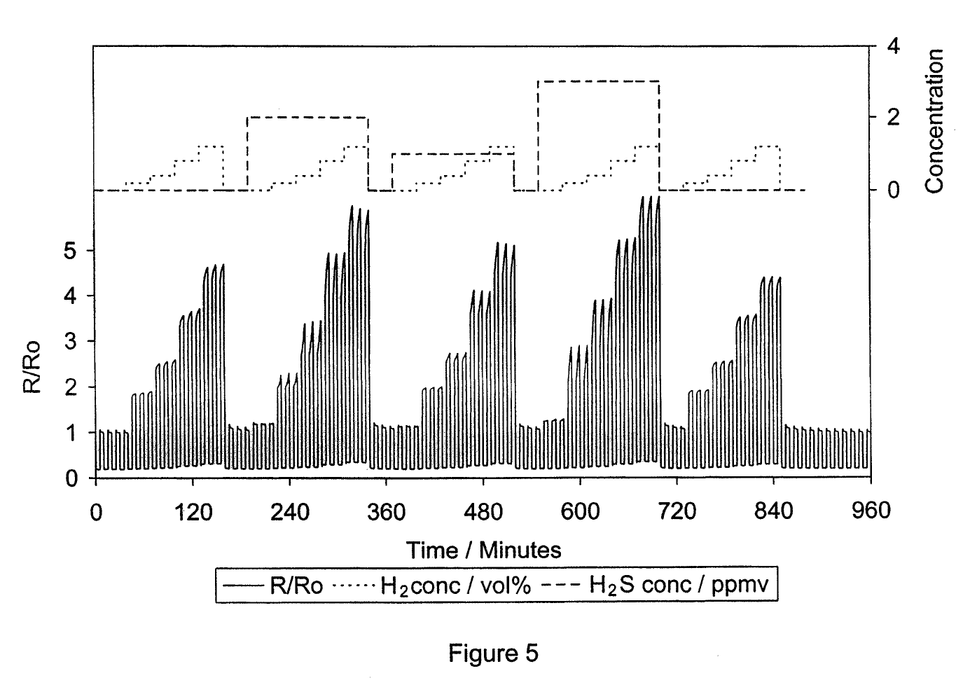

Finally, FIG. 5 shows a repeat of FIG. 1, but with the sensor temperature cycled as described above in relation to FIG. 2. Comparison of the two figures again shows that the temperature cycling approach gives reversible behaviour, removing the hysteresis present when using a single operating temperature. In particular, the sensitivities to the first and last sets of hydrogen exposures, both in air, are the same when using the temperature cycling approach, whereas they are clearly different when running at a single temperature. Also, the enhancement in hydrogen sensitivity is in proportion to the current hydrogen sulphide concentration when using temperature cycling, and is independent of previous exposure of the sensor to the gas.

Although the data presented herein is for a specific sensor and a specific target and interferent gas combination, since the conditioning effect of certain gases such as sulphur compounds is generic to most semiconducting metal oxide sensor materials, the approach here is not restricted to the particular embodiment demonstrated.

1. A method for determining the concentration of a target gas in an environment which utilises a metal oxide gas sensor having a sensitivity to a background stimulus present in the environment, said sensitivity being variable according to the concentration of the target gas, the method comprising the following steps:
(1) calibrating the sensitivity of the first sensor to the background stimulus as a function of the concentration of the target gas;
(2) determining a change in the sensitivity of the first sensor to the background stimulus in the environment, caused by the presence of the target gas in the environment; and
(3) using the change in sensitivity to the background stimulus determined in step (2) and the step of calibrating the sensitivity of the first sensor in step (1) to determine the concentration of the target gas in the environment.

2. A method according to claim 1, further comprising after step (2) resetting the sensitivity of the first sensor to the background stimulus to the sensitivity prior to exposure to the target gas, by subjecting the first sensor to elevated temperature to substantially remove the effect of the target gas on the sensitivity of the first sensor.

3. A method according to claim 2, comprising resetting the sensitivity of the first sensor by cycling the first sensor between elevated and lower temperature conditions.

4. A method according to claim 1, wherein step (2) comprises:
(a) measuring, at a measuring temperature, the sensitivity of the first sensor to the background stimulus in the environment using the first sensor shortly after exposure thereof to a temperature higher than the measuring temperature, and at which higher temperature any effect on the sensitivity of the first sensor due to the presence of the target gas is substantially cancelled;
(b) measuring, at the measuring temperature, the sensitivity of the first sensor to the background stimulus in the environment using the first sensor after a period of time has elapsed to allow the sensitivity of the first sensor to be changed by the target gas; and
(c) determining from steps (a) and (b) the change in sensitivity of the first sensor to the background stimulus caused by the presence of the target gas.

5. A method according to claim 1, wherein the background stimulus is an interferent gas, and step (2) comprises determining a change in the sensitivity of the first sensor to a known amount of the interferent gas in the environment.

6. A method according to claim 5, wherein the known amount of interferent gas is determined using a second metal oxide gas sensor of the same type as to the first sensor, and wherein the second sensor is maintained at elevated temperature, or is subjected to continuous temperature cycling.

7. A method according to claim 5, wherein the known amount of interferent gas is determined by use of another sensor which is different in type to the first sensor and which has a sensitivity to the interferent gas which is not affected by the presence of the target gas.

8. A method according to claim 5, wherein the known amount of interferent gas is determined using a second metal oxide gas sensor of the same type as the first sensor, and wherein the second sensor is subjected to intermittent flushing of its surface with a gas to substantially remove target gas present in or on the sensor.

9. A method according to claim 6, wherein the known amount of interferent gas is determined using a second metal oxide gas sensor of the same type as the first sensor, and wherein the second sensor has means for excluding the target gas from its surface, so as to prevent any change in sensitivity to the interferent gas of the second sensor caused by the target gas.

10. A method according to claim 1, wherein the background stimulus in the environment comprises an interferent gas having a concentration which varies with time, and step (1) further comprises calibrating the sensitivity of the first sensor to the interferent gas in the presence of the target gas as a function of the concentration of the interferent gas.

11. A method according to claim 1, wherein the background stimulus is an interferent gas and the target gas is present at a low level in the environment as compared to the interferent gas.

12. A method according to claim 1, wherein the target gas is selected from sulphur-containing gases.

13. A method according to claim 12, wherein the target gas is hydrogen sulphide.

14. A method according to claim 13, wherein the interferent gas is hydrogen.

15. A method according to claim 14, wherein the target gas is hydrogen sulfide and the interferent gas is hydrogen, and the environment is the inside of a fuel gas reformer.

16. A system for performing a method as in claim 1, the system comprising
a metal oxide sensor;
a temperature controller for controlling the temperature of the metal oxide sensor; and
a data processor.

17. A method of resetting the sensitivity of a metal oxide gas sensor which has an
increased or decreased sensitivity to a target gas as a result of exposure to the target gas, to the sensitivity to the target gas prior to exposure to the target gas, the method comprising subjecting the sensor to elevated temperature to substantially remove the effect of the target gas on the sensitivity of the sensor.

18. A method of resetting the sensitivity of a metal oxide gas sensor which has an increased sensitivity to an interferent gas as a result of exposure of the sensor to a target gas, to the sensitivity to said interferent gas prior to exposure to the target gas, the method comprising subjecting the sensor to elevated temperature to substantially remove the effect of the target gas on the sensitivity of the sensor.

19. A method according to claim 17, comprising cycling the sensor between elevated temperature and lower temperature conditions.

20. A method according to claim 18, comprising cycling the sensor between elevated temperature and lower temperature conditions.

21. A method according to claim 17, wherein the sensitivity of the metal oxide gas sensor is altered by the presence of hydrogen sulphide in the environment.

22. A method according to claim 18, wherein the sensitivity of the metal oxide gas is altered by the presence of hydrogen sulphide in the environment.

23. Use of a sensor which has had its sensitivity reset in accordance with a method as defined in claim 17, for determining the presence and/or concentration of hydrogen sulphide in oil field applications.

24. Use of a sensor which has had its sensitivity reset in accordance with a method as defined in claim 18, for determining the presence and/or concentration of hydrogen sulphide in oil field applications.
